(19) United States
(12) Patent Application Publication
(10) Pub. No.: US 2015/0306257 A1
(43) Pub. Date: Oct. 29, 2015

(54) PHARMACEUTICAL PREPARATION

(71) Applicant: Thomas RAMDAHL, Oslo (NO)

(72) Inventor: Thomas RAMDAHL, Oslo (NO)

(73) Assignee: ALGETA ASA, Oslo (NO)

(21) Appl. No.: 14/007,889

(22) PCT Filed: Mar. 29, 2012
(86) PCT No.: PCT/GB12/50709

§ 371 Date: Dec. 30, 2013

(30) Foreign Application Priority Data
Mar. 29, 2011 (GB) 1105298.2

(51) Int. Cl. A61K 51/06; A61K 51/12

(52) U.S. Cl. CPC A61K 51/06; A61K 51/1241

(57) ABSTRACT

A pharmaceutical preparation comprising at least one complexed alpha-emitting radionuclide and at least one polysaccharide biopolymer.

### BACKGROUND

The present invention relates to the field of endoradionuclide therapy, and in particular to alpha-endoradionuclide therapy. More specifically the present invention relates to the safety and efficacy of preparations for use in endoradionuclide therapy, to such preparations and to methods for their preparation, treatment and safe storage.

The basic principle of endo-radionuclide therapy is the selective destruction of undesirable cell types, e.g. for cancer therapy. Radioactive decay releases significant amounts of energy, carried by high energy particles and/or electromagnetic radiation. The released energy causes cytotoxic damage to cells, resulting in direct or indirect cell death. Obviously, to be effective in treating disease, the radiation must be preferentially targeted to diseased tissue such that this energy and cell damage primarily eliminates undesirable tumour cells, or cells that support tumour growth.

Certain beta-particle emitters have long been regarded as effective in the treatment of cancers. More recently, alpha-emitters have been targeted for use in anti-tumour agents. Alpha-emitters differ in several ways from beta-emitters, for example, they have higher energies and shorter ranges in tissues. The radiation range of typical alpha-emitters in physiological surroundings is generally less than 100 μm, the equivalent of only a few cell diameters. This relatively short range makes alpha-emitters especially well-suited for treatment of tumours including micrometastases, because when they are targeted and controlled effectively, relatively little of the radiated energy will pass beyond the target cells, thus minimising damage to the surrounding healthy tissue. In contrast, a beta-particle has a range of 1 mm or more in water.

The energy of alpha-particle radiation is high compared to that from beta-particles, gamma rays and X-rays, typically being 5-8 MeV, or 5 to 10 times higher than from beta-particle radiation and at least 20 times higher than from gamma radiation. The provision of a very large amount of energy over a very short distance gives alpha-radiation an exceptionally high linear energy transfer (LET) when compared to beta- or gamma-radiation. This explains the exceptional cytotoxicitiy of alpha-emitting radionuclides and also imposes stringent demands on the level of control and study of radionuclide distribution necessary in order to avoid unacceptable side effects due to irradiation of healthy tissue.

Thus, while very potent, it is important to deliver the alpha-emitting radionuclides to the tumour with little or no uptake in non-disease tissues. This may be achieved analogously to what has been shown when delivering the beta-emitting radionuclide yttrium-90 (Y-90) using a monoclonal antibody conjugated with the chelating molecule DTPA as a carrier, i.e. the clinically used radiopharmaceutical Zevalin® (Goldsmith, S. J, Semin. Nucl. Med. 40: 122-35. Radioimmunotherapy of lymphoma: Bexxar and Zevalin.). Thus, a complex of the radionuclide and the carrier-chelator conjugate is administered. Besides full length antibodies of different origins, other types of proteinaceous carriers have been described, including antibody fragments (Adams et al., A single treatment of yttrium-90-labeled CHX-A”-C6.5 diabody inhibits the growth of established human tumor xenografts in immunodeficient mice. Cancer Res. 64: 6200-8, 2004), domain antibodies (Tijink et al., Improved tumor targeting of anti-epidermal growth factor receptor Nanobodies through albumin binding: taking advantage of modular Nanobody technology. Mol. Cancer Ther. 7: 2288-97, 2008), lipochalins (Kim et al., High-affinity recognition of lanthanide(III) chelate complexes by a reprogrammed human lipocalin 2. J. Am. Chem. Soc. 131: 3565-76, 2009), affibody molecules (Tolmachev et al., Radionuclide therapy of HER2-positive microxenografts using a ¹⁷⁷Lu-labeled HER2-specific Affibody molecule. Cancer Res. 15:2772-83, 2007) and peptides (Miederer et al., Preclinical evaluation of the alpha-particle generator nuclide ²²⁵Ac for somatostatin receptor radiotherapy of neuroendocrine tumors. Clin. Cancer Res. 14:3555-61, 2008).

Decomposition or “decay” of many pharmaceutically relevant alpha emitters results in formation of “daughter” nuclides which may also decay with release of alpha emission. Decay of daughter nuclides may result in formation of a third species of nuclides, which may also be alpha emitter, leading to a continuing chain of radioactive decay, a “decay chain”. Therefore, a pharmaceutical preparation of a pharmaceutically relevant alpha emitter will often also contain decay products that are themselves alpha emitters. In such a situation, the preparation will contain a mix of radionuclides, the composition of which depends both on the time after preparation and the half-lives of the different radionuclides in the decay chain.

The very high energy of an alpha-particle, combined with its significant mass, results in significant momentum being imparted to the emitted particle upon nuclear decay. As a result, when the alpha particle is released an equal but opposite momentum is imparted to the remaining daughter nucleus, resulting in “nuclear recoil”. This recoil is sufficiently powerful to break most chemical bonds and force the newly formed daughter nuclide out of a chelate complex where the parent nuclide was situated when decomposing. This is highly significant where the daughter nucleus is itself an alpha-radiation emitter or is part of a continuing chain of radioactive decay.

Due to the recoil effects discussed above and due to the change in chemical nature upon radioactive decay, the daughter nuclides thus formed from radioactive decay of the initially incorporated radionuclide may not complex with the chelator. Therefore, in contrast to the parent nuclide, daughter nuclides and subsequent products in the decay chain may not be attached to the carrier. Thus, storage of an alpha-emitting radioactive pharmaceutical preparation will typically lead to accumulation “ingrowth” of free daughter nuclides and subsequent radionuclides in the decay chain, which are no longer effectively bound or chelated. Unbound radioisotopes are not controlled by the targeting mechanisms incorporated into the desired preparation and thus upon administration to a patient radioactive decay products will not be directed to tumour tissue and will distribute in the body, leading to undesirable irradiation of healthy tissues.

Since most radioisotopes need to be generated and purified in dedicated production facilities, a certain storage period between formation and administration is inevitable, and it is desirable that a pharmaceutical preparation be as stable and safe to storage as is practicable. A significant problem with past methods has been to administer a reproducible composition of a targeted alpha-radionuclide, which does not contain variable amounts of non-targeted alpha-radionuclides in relation to the targeted amount.

Although the decay of the desired nuclide during the storage period can be calculated and corrected for, this does not avoid the build-up of un-targeted daughter products which can render the composition more toxic and/or reduce the safe storage period and/or alter the therapeutic window in undesirable ways. In addition, it would thus be of benefit for the compositions to be safe for storage or to provide a method by which a stored composition can be assured as safe.

The events following decomposition of thorium-227 may be considered as an illustration of the challenge. With a half-life of about 18.7 days thorium-227 decomposes into radium-223 upon release of an alpha-particle. Radium-223 in turn has a half-life of about 11.4 days, and decomposing into radon-219, giving rise to polonium-215, which gives rise to lead-211. Each of these steps gives rise to alpha-emission and the half-lives of radon-219 and polonium-215 are less than 4 seconds and less than 2 milliseconds, respectively. The end result is that the radioactivity in a freshly prepared solution of e.g. chelated thorium-227 will increase over the first 19 days, and then start to decrease. Clearly the amount of thorium-227 available for being targeted to a tumor is constantly decreasing, and thus the fraction of the total radioactivity deriving from thorium-227 is dropping during these 19 days, when an equilibrium situation is reached. If daughter nuclides could be specifically removed in a simple procedure, only the amount of thorium-227 would have to be considered, and the therapeutic window—the relation between therapeutic effect and adverse effects would be unrelated to the time of storage.

A further aspect of the ingrowth of daughter nuclides in a pharmaceutical solution relates to the radiolysis of a carrier such as an antibody. Since radiolysis depend on the concentration of both the carrier and the radioactivity, the increase of radioactivity in the solution resulting from occurrence of daughter nuclides will put a limit on the acceptable starting radioactivity in relation to the desired shelf life. Thus, to interfere with the radioactivity deriving from daughter nuclides from reaching the carrier would be beneficial in terms of shelf-life at any given starting concentration of radioactivity.

Thus, there is considerable ongoing need for improved radiotherapeutic compositions (particularly for alpha-emitting radionuclides), and procedures for making a solution ready for injection whose biological effects may be reproducibly assessed, without having to consider ingrown radionuclides formed in the radioactive decay chain. Furthermore, there is a need for radiotherapeutic methods and kits allowing facile preparation of a final radioactive formulation under sterile conditions directly prior to administration to a patient.

### DETAILED DESCRIPTION

The present invention relates to compositions, methods and procedures for removal of cationic daughter nuclides from a radiopharmaceutical preparation containing a parent radionuclide stably chelated to an entity also containing a targeting moiety, i.e. the parent radionuclide is complexed. In particular, the present inventors have surprisingly established that daughter radionuclides may be safely and reliably captured onto pre-formed biocompatible and biodegradable hydrogel particles, or other structures. The radionuclides are particularly alpha-emitting radionuclides or generators for alpha-emitting radionuclides. The final therapeutic formulations obtained from application of the invention are suitable for use in the treatment of both cancer and non-cancerous diseases.

Alternative phrased; the invention provides a composition allowing continuous removal of radioactive daughter nuclides up until immediately before distribution in vivo, where ingrown radioactive decay products are removed. This leads to minimal co-administration of daughter nuclides and hence minimizing radiation dose and radiation damage on normal and non-target tissues.

Thereby, only the concentration and the half-life of the parent radionuclide and of daughter nuclides formed in vivo have to be taken into consideration when calculating the radioactive dose obtained by the patient. Most importantly this leads to a reproducible situation with regard to the relation between efficacy and adverse effects. Thus, the available therapeutic window will not change with storage time of the pharmaceutical preparation.

Phrased differently; by applying the invention the relation between desired anti-tumour effects and adverse effects may be directly related to the measured concentration of the primary nuclide and becomes independent of the time of storage of the pharmaceutical preparation. In situations where the concentration of the primary alpha-emitting radionuclide may be determined by measuring one or more parallel emissions of gamma radiation, sufficiently separate from and gamma emission from the daughter emissions, this may be performed using standard equipment at the radiopharmacy. In fact, if the starting material is pure, the relevant dose of the pharmaceutical preparation will depend only on the time after manufacturing and may be tabulated. In principle there is no need for further measurements at the clinic and the corresponding radiopharmaceutical could be handled in analogy to any other toxic pharmaceutical (although such a procedure would counter current practice, which is based on the fact that radioactivity can be easily measured). The enablement of this new and simplified procedure for clinical handling of targeted alpha-emitting radiotherapeutics is an important aspect of the invention.

Another aspect of the invention is to provide a pharmaceutical preparation where daughter nuclides are continuously removed, whereby the rate of radiolysis e.g. of the carrier, chelating moiety and/or targeting moiety, of the radiopharmaceutical is not increasing over time of storage. This is beneficial in terms of shelf-life.

It has been established by the present inventors that biocompatible and biodegradable hydrogel particles that can be formed from polysaccharide biopolymers, will, to a high extent, retain cationic daughter nuclides after decay of the parent nuclide. This provides a considerable advantage in the preparation and delivery of high quality radiopharmaceuticals which can be prepared some time prior to administration but delivered with a relatively low level of contamination from uncomplexed daughter radionuclides.

In a first aspect, the invention therefore provides a pharmaceutical preparation comprising at least one complexed alpha-emitting radionuclide and at least one polysaccharide biopolymer. Preferably said polysaccharide biopolymer will absorb or be capable of absorbing uncomplexed ions. In particular said polysaccharide biopolymer will absorb or be capable of absorbing uncomplexed ions resulting from the radioactive decay of the complexed alpha-emitting radionuclide. These may be the direct daughter nuclides or those further down the radioactive decay chain.

It is particularly important that the solution level of uncomplexed radioactive isotopes resulting from the radioactive decay chain of the complexed alpha-emitting radionuclide be kept low and thus it is preferably that the polysaccharide biopolymer will absorb or be capable of absorbing these. As used herein the term “daughter isotope” is used to indicate both the direct decay product of a radioisotope and also any isotope further down the decay chain that may result from one or more further decays (where context permits). Similarly, isotopes described as “resulting from the decay chain” of a radionuclide include any isotopes which may be formed as a result of that decay of that radioisotope and also any further daughter products which may result from subsequent decays in the chain.

The inventors have surprisingly established that appropriate biopolymers are highly effective in absorbing unwanted uncomplexed ions from a solution of complexed radioisotope. Consequently, in a second aspect the present invention provides a method for generating an injectable solution of at least one complexed alpha-emitting radionuclide, said method comprising contacting a pharmaceutical preparation of said least one complexed alpha-emitting radionuclide with at least one polysaccharide biopolymer and subsequently separating said solution of at least one complexed alpha-emitting radionuclide from said at least one polysaccharide biopolymer. Typically the separation comprises will be by means of filtration, preferably sterile filtration. This is particularly appropriate as the final step prior to administration.

In a corresponding aspect, the present invention further provides a method for the removal of at least one uncomplexed radionuclide from a pharmaceutical preparation comprising a solution of at least one complexed alpha-emitting radionuclide, said method comprising contacting said pharmaceutical preparation with at least one polysaccharide biopolymer. Such a method will preferably also comprise separating said solution from said polysaccharide biopolymer. Any suitable separation method, such as any of those described herein may be used in this and any appropriate aspect.

In a further aspect the invention further provides the use of at least one polysaccharide biopolymer for the removal of at least one uncomplexed radionuclide from a pharmaceutical preparation comprising a solution of at least one complexed alpha-emitting radionuclide. Such a use will typically be by contacting said polysaccharide biopolymer with said pharmaceutical preparation and subsequently separating said polysaccharide biopolymer from said solution (e.g. by filtration).

Since the pharmaceutical preparations may be provided directly in an administration device (such as a syringe) ready for administration, the invention further provides, in another aspect, an administration device comprising a pharmaceutical preparation as described herein. Such a device will typically be equipped with a method for separation of the biopolymer from the solution prior to or during administration. Such a device may be, for example, a filter, such as a sterile filter. Syringe-filters are appropriate for syringes and similar devices.

Since the present invention is highly suitable for final purification of a pharmaceutical preparation prior to administration, the invention additional provides, in a further aspect, a kit for the preparation of an injectable solution, said kit comprising least one polysaccharide biopolymer and at least one solution of a complexed alpha-emitting radionuclide. Such a kit will generally comprise a pharmaceutical preparation as described herein. Optionally and preferably the kit will additionally comprise a means for separating the solution component of the kit from the biopolymer. A filter device is preferred in this respect. The kit of the invention may comprise an administration device, which may be a pre-filled administration device as described herein.

The injectable solutions formed or formable by the methods and uses of the invention are highly suitable for use in therapy, particularly for use in the treatment of hyperplastic or neoplastic disease.

As used herein, the term “pharmaceutical preparation” indicates a preparation of radionuclide with pharmaceutically acceptable carriers, excipients and/or diluents. However, a pharmaceutical preparation may not be in the form which will ultimately be administered. For example, a pharmaceutical preparation may require the addition of at least one further component prior to administration and/or may require final preparation steps such as sterile filtration. A further component can for example be a buffer solution used to render the final solution suitable for injection in vivo. In the context of the present invention, a pharmaceutical preparation may contain significant levels of uncomplexed radionuclides resulting from the radioactive decay chain of the desired radionuclide complex which will preferably be removed to a significant degree by a method according to the present invention before administration. Such a method may involve the continuous absorption of such uncomplexed radionuclides over a significant part of the storage period of the preparation, or may take place at the final stage, immediately before administration. A pharmaceutical preparation may comprise at least one biopolymer component as described herein. Any metal ion bound to or within such a polymer, although contained within the preparation, are not considered to be “in solution” and to be “uncomplexed” in contrast to the parent radionuclide in that preparation when described herein, i.e. is not encompassed in the expression “solution concentration”.

In contrast to a pharmaceutical preparation, an “injectable solution” or “final formulation” as used herein indicates a medicament which is ready for administration. Such a formulation will also comprise a preparation of complexed radionuclide with pharmaceutically acceptable carriers, excipients and/or diluents but will additionally be sterile, of suitable tonicity and will not contain an unacceptable level of uncomplexed radioactive decay products. Such levels are discussed in greater detail herein. Evidently, an injectable solution will not comprise any biopolymer component, although such a biopolymer will preferably have been used in the preparation for that solution as discussed herein.

The invention provides a simple method for purification and preparation of a sterile final formulation of a radioactive preparation ready for administration, using structures of at least one polysaccharide biopolymer to capture unwanted radioactive decay products and a rapid separation of the loaded radioactive particles from the solution immediately prior to administration to a patient. The separation may be achieved by the sterile filtration performed as the final formulation is drawn into the syringe, subsequently to be used for administration to the patient.

Implemented as described, the invention provides a simple kit (as described herein) for purification and final formulation of a radioactive medicament for use in therapy. The kits of the invention may for example include a vial with the pharmaceutical solution, a sterile filter and a syringe. The components of the kit may be separate or coupled together into one unit.

The invention provides for the use of the procedure for preparation of a final formulation for injection, for example using components provided as a kit. The procedure of any of the methods and/or uses of the invention may include an incubation step where the pharmaceutical preparation is mixed for example by gentle shaking, to enable optimal capture of daughter nuclides by the polysaccharide biopolymer provided as particles or as coating on the inside surface of the vial.

The pharmaceutical preparations of the invention, wherein contact is made between a solution of a complexed radionuclide and a biopolymer, will desirably have a low concentration of uncomplexed metal ions, particularly a low concentration of uncomplexed radioactive metal ions in the solution. Typically, for example, the solution concentration of uncomplexed ions of radioisotopes (such as alpha emitting radioisotopes) should preferably contribute no more than 10% of the total count of radioactive decays per unit time (from the solution), with the remainder being generated by decay of a complexed alpha radionuclide. This will preferably be no more than 5% of the total count and more preferably no more than 3%. It will be evident that since the biopolymer is serving to capture the uncomplexed radioactive daughter isotopes, the radioactive count of radioactive decays per unit time from the biopolymer and entrained nuclides may be a great deal higher. This biopolymer is, however, separated from the solution component prior to administration. Thus, correspondingly, the methods and uses of the invention may comprise the step of separating a solution component comprising a complexed alpha-emitting radioisotope from a biopolymer component containing entrained uncomplexed radioactive ions, whereby to leave a solution having a concentration of uncomplexed ions of radioisotopes (such as alpha emitting radioisotopes) which contributes no more than 10% of the total count of radioactive decays per unit time. This will preferably be no more than 5% of the total count and more preferably no more than 3%.

Similarly, since the solution concentration of uncomplexed radionuclides is low, a pharmaceutical preparation as described herein may typically have solution concentration of uncomplexed ions resulting from the radioactive decay chain of at least one complexed alpha-emitting radionuclide which is no greater than 10% (by mol/litre) of the solution concentration of said least one complexed alpha-emitting radionuclide. This will preferably be no more than 5% of the total count and more preferably no more than 3%.

Furthermore, since the solution concentration of uncomplexed radionuclides is low, with the bulk of such radionuclides captured by the biopolymer, a pharmaceutical preparation as described herein may typically have a radioactive count generated from decay uncomplexed ions in solution (especially those resulting from the radioactive decay chain of at least one complexed alpha-emitting radionuclide) which is no more than 10% of the count generated from decay of uncomplexed ions captured by said at least one polysaccharide biopolymer. This will preferably be no more than 5% of the total count and more preferably no more than 3%.

One additional advantage of the various aspects of the present invention is that the biopolymer may be used to maintain a low level of uncomplexed radionuclides in the solution during the storage and transportation period which will elapse between generation of a radiopharmaceutical and its administration. Thus, a pharmaceutical preparation as described herein will preferably have a solution concentration of uncomplexed radionuclidic ions which contributes no more than 10% of the total radioactive decay count (decays per unit time) of the solution portion of the pharmaceutical preparation for a period of at least 2 half-lives of the longest lived of the complexed alpha-emitting radionuclides. This will preferably be at least 3 of the specified half-lives, more preferably at least 4 of said half-lives/

In the pharmaceutical preparations of the invention is at least one complexed alpha-emitting radionuclide. Generally, such nuclides will be of nuclear mass of at least 100 and will have a half-life of between 4 hours and 1 year, preferably between 1 day and 60 days. Preferable complexed apha radionuclides include at least one complexed alpha-emitting radionuclide selected from ²²⁷Th, ²²³Ra, ²²⁵Ac. The most preferred alpha-emitter is ²²⁷Th.

In the pharmaceutical preparations of the invention and correspondingly in the resulting solutions for injection, at least one alpha-emitting radionuclide is complexed by means of a suitable chelating entity. Many suitable chelators are known for the various suitable alpha-emitting radionuclides, such as those based on on DOTA (1,4,7,10-tetraazacyclododecane-1,4,7,10-tetraacetic acid) and other macrocyclic chelators, for example containing the chelating group hydroxy phthalic acid or hydroxy isophthalic acid, as well as different variants of DTPA (diethylene triamine pentaacetic acid), or octadentate hydroxypyridinone-containing chelators. Preferred examples are chelators comprising a hydroxypyridinone moiety, such as a 1,2 hydroxypyridinone moiety and/or a 3,2-hydroxypyridinone moiety. These are very well suited for use in combination with ²²⁷Th.

In the pharmaceutical preparations of the invention and correspondingly in the resulting solutions for injection the at least one complexed alpha-emitting radionuclide is preferably bound to at least one targeting moiety. Many such moieties are well known in the art and any suitable targeting moiety may be used, individually or in combination. Suitable targeting moieties include poly- and oligo-peptides, proteins, DNA and RNA fragments, aptamers etc. Preferable moieties include peptide and protein binders, e.g. avidin, strepatavidin, a polyclonal or monoclonal antibody (including IgG and IgM type antibodies), or a mixture of proteins or fragments or constructs of protein. Antibodies, antibody constructs, fragments of antibodies (e.g. Fab fragments, single domain antibodies, single-chain variable domain fragment (scFv) etc), constructs containing antibody fragments or a mixture thereof are particularly preferred.

In addition to the various components indicated herein, the pharmaceutical preparations may contain any suitable pharmaceutically compatible components. In the case of radiopharmaceuticals, these will typically include at least one stabiliser. Radical scavengers such as ascorbate and/or citrate are highly suitable. Serum albumin, such as BSA, is also a suitable additive, particularly for protection of protein and/or peptide components such as antibodies and/or their fragments.

In the methods and uses of the present invention, the contacting between the solution part of the pharmaceutical preparation and the biopolymer may take place continuously from the time or preparation of the pharmaceutical until shortly before its administration. This is the most preferred method. Alternatively, however, the contacting may be carried out for only a short period immediately before the solution is withdrawn for administration. Thus, in one preferred embodiment, said contacting takes place for greater than 50% of the storage period between preparation of said pharmaceutical preparation and separating said solution of at least one complexed alpha-emitting radionuclide from said at least one polysaccharide biopolymer.

In an alternative embodiment, said contacting takes place for no more than 8 hours (e.g. no more than 3 hours), preferably no more than 1 hour prior to separating said solution of at least one complexed alpha-emitting radionuclide from said at least one polysaccharide biopolymer.

In all aspects of the present invention including the methods, uses, kits and devices of the invention, provision is preferably made for the separation of the solution containing at least one complexed alph-radionuclide from the polysaccharide biopolymer. Where the biopolymer is a surface coating (e.g. on a vial or the well of a plate) then this may be carried out simply by withdrawing or decanting the solution. Preferably, however, the separation may take place by filtration. Preferably such filtration will be sterile filtration and will thus also generate a sterile solution suitable for injection. Correspondingly, the kits of the invention may optionally and preferably additionally comprising a filter (e.g. of porse size 0.45 μm or of pore size of about 0.22 μm). In all cases filtration through a filter of pore size no larger than 0.45 μm, preferably no larger than 0.22 μm is preferred.

All aspects of the invention relate to structures of at least one polysaccharide biopolymer having the property of binding at least one radionuclide.

The polysaccharide biopolymer structures suitable for use in all aspects of the invention may be of any shape and size providing that they provide sufficient surface area that they are capable of binding an effective amount of radionuclide.

Typically, the particles will be approximately spherical in shape as this provides a large and regular surface for binding of the radionuclide, and eases manufacture. Other particle shapes which can achieve sufficient surface area are suitable, however, including, for example ellipsoidal, rod-shaped and plate-shaped particles, fibres, sheets, threads and woven materials. The polysaccharide biopolymer may also be used for coating the inside surface of the vial, facilitating the preparation of the final formulation for injection.

To facilitate sterile filtration the size of the particles or structures should not be small enough to enter the filter used, normally 0.22 or 0.45 μm cut-off for a spherical particle. Thus, in the manufacturing and handling of the particles or other structures care has to be taken to avoid inclusion of shapes and sizes that will enter a pharmaceutically acceptable sterile filter. This requirement is more important than obtaining a large surface area. The minimum size of the smallest dimension of a particle is thus preferably at least 0.4 μm, more preferably at least 0.5 μm, most preferably at least 10 μm. It is preferable that not more than 1% of the particles have any dimension smaller than the appropriate cut-off value.

The particles used in the method encompassed by the invention may be homogeneous or inhomogeneous, where the homogeneity refers to the concentration gradient of the polysaccharide biopolymer from the interior to the exterior of the particle. A homogenous polysaccharide biopolymer particle has a regular distribution of polysaccharide biopolymer throughout the particle cross-section, such that the concentration gradient is substantially zero. As used herein, “concentration gradient” is the concentration of polysaccharide biopolymer in a particle as a function of distance from the centre of the particle towards the surface of the particle. An inhomogeneous polysaccharide biopolymer particle has an irregular distribution of polysaccharide biopolymer throughout the particle. This is generally such that the concentration of polysaccharide biopolymer is greater towards the surface of the particle than at the core of the particle. Homogeneous and inhomogeneous spheres are illustrated in FIG. 1. Both homogeneous and inhomogeneous particles are effective for the purposes of the invention.

The polysaccharide biopolymer particles of the invention are preferably cross-linked using metal ions. Preferred cross-linkable polysaccharides are alginates, which may be cross-linked with any suitable cations. Alginate is a structural polysaccharide found in brown algae, comprising up to 40% of the dry matter. Its main function is to give strength and flexibility to the algal tissue. Alginate is an unbranched binary copolymer of 1-4-linked β-D-mannuronic acid (M) and α-L-guluronic acid (G) residues (shown in FIG. 2). The relative amount of the two uronic acid monomers and their sequential arrangement along the polymer chain vary widely, depending on the origin of the alginate. The uronic acid residues are distributed along the polymer chain in a pattern of blocks, where homopolymeric blocks of G residues (G-blocks), homopolymeric residues of M residues (M-blocks) and blocks with alternating sequences of M and G units (MG-blocks) co-exist. Thus the alginate molecule cannot be wholly described by the monomer composition alone. NMR characterisation of the sequence of M and G residues in the alginate chain is needed in order to calculate the average block lengths, and suitable methods are well known in the art. It has also been shown by NMR analysis that alginate has no regular repeating unit. The functional properties of alginate are primarily influenced by the G content, the average number of Gs in a G-block length and the molecular weight.

Alginate forms gels with most di- and multivalent cations, although calcium is most widely used. Monovalent cations and the divalent Mg²⁺ ions do not induce gelation (I. W. Sutherland, “Alginates”, Biomaterials; Novel Materials from Biological Sources, D. Byrom, ed., New York, 1991, pp. 309-331). The gelling reaction occurs when divalent cations take part in interchain binding between G-blocks within the alginate molecules, giving rise to a three-dimensional network in the form of a gel. Gelation with, for example, calcium ions results in the instantaneous formation of heat-stable gels that can be formed and set at room temperature and at physiological pHs. The gel strength will depend on the guluronate content and also on the average number of G-units in the G-blocks (N_{G>1}). In addition, using alginates with increasing molecular weights will also increase the strength of the gel, at least up to a certain limit of molecular weight. It has been observed by the present inventors that alginate gels effectively bind many metal cations. This is of advantage in allowing capture of a variety of radionuclides, but it is of crucial significance where decay of the desired radionuclide is followed by a continuing chain of radioactive decays because a variety of chemically distinct radioisotopes will then be present. Furthermore, these additional radioisotopes may contribute to radiolysis of the targeting moiety of the complex with the parent radionuclide, unless controlled, for example by being captured by the alginate and removed from solution.

It has been shown that the properties of an alginate gel strongly depend upon the method of preparation. When a gel bead is formed by diffusion of calcium ions into droplets of alginate solution, a non-uniform distribution of polymer in the bead is obtained. This can be explained by differences in the diffusion rate of the gelling ions into the bead relative to the diffusion rate of the alginate molecules towards the gelling zone (G. Skjåk-Braek, O. Smidsrød & H. Grasdalen, “Inhomogeneous polysaccharide ionic gels.” Carbohydr. Res., 10, 1989, pp. 31-54). Another factor effecting homogeneity is the presence of non-gelling ions like Na⁺ or Mg²⁺. Such ions will compete with the gelling ions during the gelling process, resulting in more homogenous beads. More homogeneous beads will also be mechanically stronger and have a higher porosity than more inhomogeneous beads. For example, adding sodium chloride together with calcium chloride results in the formation of a more homogeneous gel bead. Maximum homogeneity is reached with a high molecular weight alginate gelled with high concentrations of both gelling and non-gelling ions.

For the present application, mechanical strength is preferred over higher porosity.

In a further embodiment applicable to all aspects of the invention, the polysaccharide biopolymer may optionally be coated with at least one material to maintain its integrity and/or to reduce disintegration due to radiolysis.

A further embodiment of the invention is application of particles or other structures composed of mixed polymers, obtained by combining alginate with another polymer that provides further structural integrity and/or shape, and to which a protein such as an antibody carrying the therapeutic nuclide is not adhering. Polymers previously used in pharmaceutical preparations are preferred, including polyethylenglycol and branched structures thereof.

In one aspect, the invention provides a pharmaceutical preparation comprising particles of at least one polysaccharide biopolymer having the ability to bind radiometals and at least one pharmaceutically acceptable excipient or carrier. Pharmaceutically tolerable carriers and excipients are well-known to a person skilled in the art and may include, for example, salts, sugars and other tonicity adjusters, buffers, acids, bases and other pH adjusters, viscosity modifiers, colourants etc.

Especially preferred are isotonic saline, but any other liquid carrier or carrier mixture that is physiologically acceptable and compatible with the radionuclide carrier-chelate conjugate complex can be used. Many such liquid and gel carriers or carrier systems are known by persons skilled in the art of preparing pharmaceutical preparations.

The injectable solution obtained from compositions or pharmaceutical formulations of the invention are suitable for treatment of a range of diseases and are particularly suitable for treatment of diseases relating to undesirable cell proliferation, such as hyperplastic and neoplastic diseases. For example, metastatic and non-metastatic cancerous diseases such as small cell and non-small cell lung cancer, malignant melanoma, ovarian cancer, breast cancer, bone cancer, colorectal cancer, pancreatic cancer, bladder cancer, cervical cancer, sarcomas, lymphomas, leukemias, tumours of the prostate, and liver tumours are all suitable targets. The “subject” of the treatment may be human or animal, particularly mammals, more particularly primate, canine, feline or rodent mammals.

Other aspects of the invention are the provision of a composition according to the invention, or alternatively the use of a composition according to the invention in the manufacture of a medicament for use in therapy. Such therapy is particularly for the treatment of diseases including those specified herein above. By “treatment” as used herein, is included reactive and prophylactic treatment, causal and symptomatic treatment and palliation.

Use of the medicament resulting from the invention in therapy may be as part of combination therapy, which comprises administration to a subject in need of such treatment an injectable solution according to the invention and one or more additional treatments. Suitable additional treatments include surgery, chemotherapy and radiotherapy (especially external beam radiotherapy).

Combination therapy is a particularly preferred embodiment of the present invention and may be executed in a simultaneous, sequential or alternating manner, or any combination thereof. Thus, a combination treatment may comprise one type of treatment followed by one or more other types of treatment, wherein each type of treatment may be repeated one or more times. One example of simultaneous combination therapy is chemotherapy combined with the administration of a final formulation according to the present invention at the same point in time (either by the same or by a different method of administration). Such combination treatment may be combined with sequential therapy by starting simultaneous treatment, for instance, after a tumour has been removed surgically. The combination therapy may be repeated one or more times as needed based on the patient's condition.

An example of alternating combination therapy could be chemotherapy in one or more treatment periods alternating on different days or weeks with the administration of the final formulation of the invention.

In a further aspect the invention encompasses apparatus or kit, for example, a pre-loaded device for performing of the method of the invention, such as a vial, syringe or other vessel loaded with the composition of the invention, connected over a sterile filter to a syringe or other administration device to be used to administer the final formulation. Thus, a typical apparatus includes a kit for instant purification by the composition of the invention, for example a kit comprising a quantity of polysaccharide biopolymer particles in a solution of the radioactive preparation of carrier-attached and chelated primary radionuclide and ingrown daughter nuclides stored in a vessel, and preferably, an administration device, such as a syringe. The radioactive preparation is mixed, incubated and the final formulation isolated e.g. by sterile filtration, centrifuge spinning or gravity, or by a combination of these unit operations.

As will be shown in the Examples, close to 100% of ingrown ²²³Ra²⁺, ²¹¹Pb²⁺ and ²¹¹Bi³⁺ nuclides in a preparation of ²²⁷Th⁴⁺ are bound to alginate gel beads.

The Injectable solutions formed and formable from the pharmaceutical compositions of the invention and those formed by use of the kits of the invention will evidently form a further aspect of the invention. Such solutions may be, for example an injectable solution comprising a solution of at least one complexed alpha-emitting radionuclide and at least one pharmaceutically acceptable carrier or diluent wherein the solution concentration of any uncomplexed ions resulting from the radioactive decay chain of said least one complexed alpha-emitting radionuclide is no greater than 10% of the solution concentration of said least one complexed alpha-emitting radionuclide. Such solutions may be formed or formable by any of the methods of the invention and/or by removal of the biopolymer component from any of the compositions of the invention (eg by filtration).

### EXAMPLES

### Example 1

### Capture of Radionuclides onto Alginate Gel Beads

In this example, a solution of ²²³Ra²⁺ in equilibrium with its daughter nuclides ²¹¹Pb²⁺ and ²¹¹Bi³⁺ was used.

A known amount of pre-formed alginate gel beads are washed successively to remove excess gelling cations using, for example, purified water, 0.9% NaCl solution (also called saline), or phosphate-buffered saline (also called PBS). A neutralizing buffer is suggested to avoid any detrimental effects of low pH (high acid concentration) if the radionuclide is introduced in an acidic solution.

Radium-223 as ²²³RaCl₂ solution is diluted in an aliquot of PBS. The radioactive content (measured as Bq/mL) of this solution is determined by appropriate means. Such means include, but are not limited to, gamma spectroscopy using high performance germanium detector (HPGe, EG&G Ortec, GEM 15-P germanium detector) or sodium iodide scintillation detector (NaI, Perkin Elmer, Wizard 1480), to name two techniques.

A known amount (weight) of alginate gel beads is then mixed with a known amount of radioactivity (Bq/mL). For binding of ²²³Ra²⁺, room temperature conditions, intermittent shaking, and 30-60 minutes incubation conditions are sufficient to bind all available ²²³Ra²⁺ and its daughter nuclides.

### Example 2

### Binding of ²²³Ra²⁺ and Daughter Nuclides to Calcium Cross-Linked Homogeneous and Inhomogeneous Alginate Gel Beads

The method of gelling alginate can affect the internal structure of the alginate gel. If one evaluates the alginate concentration through the midsection of an alginate gel bead, there will be a difference between gels made with sodium chloride present in the gelling solution to gels made with mannitol present in the gelling solution. Sodium ions represent a non-gelling cation that will compete with calcium during the gelling reaction. This effectively slows down the movement of alginate within the forming gel bead. If the alginate molecules don't move, then the resulting gel will have a uniform alginate concentration throughout—a homogeneous gel. However, if there are no other ions that can compete or interfere with calcium binding, then an inhomogeneous gel will form. Here the calcium diffuses into the alginate droplet while alginate inside the droplet is moving towards the gelling zone. This creates an alginate gradient through the gel bead resulting in a higher alginate concentration along the outer section of the bead and a lower alginate concentration in the center of the bead (FIG. 1). A higher alginate concentration along the outer section of the gel bead gives strength to the bead as well as providing a larger number of binding sites available for binding further ions, including radionuclidic ions.

FIG. 3 shows the result from an experiment where homogeneous calcium cross-linked alginate gel beads were made and then incubated in a solution of ²²³RaCl₂.

### Example 3

### Capture of Daughters in the Presence of Thorium-227

Calcium cross-linked, inhomogeneous alginate gel beads were incubated with a solution of Thorium-227 as ²²⁷Th⁴⁺. As can be seen in FIG. 4, 100% of the ²²³Ra²⁺, ²¹¹Pb²⁺ and ²¹¹Bi³⁺ produced during radioactive decay of ²²⁷Th bound to pre-formed alginate gel beads following 1 hour of incubation, whereas 30% of the ²²⁷Th⁴⁺ bound (FIG. 4).

A second experiment was performed, showing that the capture is very rapid, and essentially complete within 5 minutes (FIG. 5).

### Example 4

### Capture of Radioactive Daughter Nuclides from a Preparation of Th-227

A preparation of a ²²⁷Th-labelled monoclonal antibody with daughter nuclides (total activity 300 kBq in 1 mL PBS) was added inhomogeneous calcium or strontium cross-linked alginate gel particles/beads (500 mg), followed by incubation for 1 hour at room temperature. The radionuclide binding efficiency of the alginate gel beads was assessed by measuring the amount of each radionuclide in the incubation solution (S+beads), in half the supernatant (½S) and in the residual fraction containing the alginate gel beads (½S+beads). Measurements were conducted on a high performance germanium detector (HPGe, EG&G Ortec GEM 15-P). The following equation was used to calculate the fraction binding to the gel beads:

$$\% alginate gel bead binding = \frac{( \frac{1}{2} S + beads ) - ( \frac{1}{2} S )}{( S + beads )} \times 100 \%$$

The radionuclides ²²³Ra²⁺, ²¹¹Pb²⁺ and ²¹¹Bi³⁺ were removed from the ²²⁷Th-mAb with similar high efficiency in calcium and strontium cross-linked inhomogeneous alginate gel beads (Table 1).

TABLE 1
Fraction captured by the gel beads.

| | ²²³Ra²⁺ | ²¹¹Pb²⁺ | ²¹¹Bi³⁺ |
|---|---|---|---|
| Ca-beads | 99% | 91% | 81% |
| Sr-beads | 96% | 91% | 96% |

The fraction bound to the gel is slightly underestimated as the volume of the beads is not considered in the calculation.

### Example 5

### Removal of Radioactive Daughter Nuclides from a Preparation of Th-227

A preparation of a ²²⁷Th-labelled monoclonal antibody with daughter nuclides (total activity 65-110 kBq in 1 mL PBS) was added inhomogeneous strontium cross-linked alginate gel particles (25-500 mg), followed by incubation for 5-120 minutes at room temperature. The radionuclide binding efficiency of the alginate gel particles was assessed by measuring the amount of each radionuclide found in the incubation solution (S+beads), in half the supernatant (½S) and in the residual fraction containing the alginate gel particles (½S+beads). Measurements were conducted on a high performance germanium detector (HPGe, EG&G Ortec GEM 15-P). The fraction of alginate gel binding nuclide was calculated as in Example 4, slightly underestimating the true value, as the volume of the beads is not considered in the calculation.

The results are summarized in FIGS. 6 and 7. The graphs show that most of the ²²³Ra²⁺, ²¹¹Pb²⁺ and ²¹¹Bi³⁺ are removed in less than 5-15 minutes from all ²²⁷Th-mAb preparations added ≧25 mg alginate gel beads. An average loss of approximately 30% ²²⁷Th-associated radioactivity was observed. However, the recovery of ²²⁷Th increased to ≧95% after washing the alginate gel beads 3 times with 1 mL 0.9% NaCl. The radiochemical purity of ²²⁷Th was not investigated, hence part of the loss could be free ²²⁷Th⁴⁺ binding to the gel.

### Example 6

### Comparison of Alginate Gel Beads and Size Exclusion Chromatography for Removal of Unwanted Daughter Nuclides from a Preparation of Thorium-227

Preparations of radiolabelled proteins, peptides, antibodies or other high molecular weight compounds may be purified using size exclusion chromatography, removing free radionuclides from the solution. However, removal of radioactive cations from a preparation may be performed more rapidly and with less handling of the radioactive preparation using alginate gel particles/beads.

A preparation with ²²⁷Th-mAb and its radioactive daughter nuclides ²²³Ra²⁺, ²¹¹P^{b2+} and ²¹¹Bi³⁺ was purified using alginate gel beads and traditional size exclusion chromatography on a NAP-5 column (GE Healthcare Life Sciences). One part of the ²²⁷Th-mAb solution (5 MBq in 0.4 mL 0.5 M NaOAc-buffer, pH 5.5) was incubated with strontium cross-linked alginate gel beads (0.3 g) for 15 minutes at room temperature, while the other part was purified using the standard procedure for a NAP-5 column. The amounts of each radionuclide present in the different fractions were measured using a high performance germanium detector (HPGe, EG&G Ortec GEM 15-P).

TABLE 2
Fractions captured and recovered by alternative methods.

| | ²²³Ra²⁺ | ²¹¹Pb²⁺ | ²¹¹Bi³⁺ |
|---|---|---|---|
| Alginate gel beads: | | | |
| Sr-alginate gel beads (%) | 97.5 | 97.5 | 96.9 |
| ²²⁷Th-mAb product (%) | 2.5 | 2.5 | 3.1 |
| NAP-5 purification: | | | |
| NAP-5 column (%) | 98.6 | 98.7 | 97.8 |
| ²²⁷Th-mAb product (%) | 1.4 | 1.3 | 2.2 |

The results are summarized in Table 2, showing 97-99% removal of the ²²³Ra²⁺, ²¹¹Pb²⁺ and ²¹¹Bi³⁺ with both purification methods. Hence both methods are very efficient for removal of the radioactive daughter nuclides of ²²⁷Th. The fraction of alginate gel binding nuclide was calculated as in Example 4, slightly underestimating the true value, as the volume of the beads is not considered in the calculation. This could explain the slightly lower fraction captured using the gel beads compared to the standard procedure.

Purification by alginate gel beads is less time consuming and very easy, only involving the use of simple, standard laboratory equipment:
1) There were no preparations prior to the alginate gel bead purification.
2) The ²²⁷Th-mAb preparation was simply added to the alginate gel beads, followed by gentle mixing and incubation for 15 minutes at room temperature.
3) Alginate gel beads were allowed to precipitate and the purified ²²⁷Th-mAb solution was carefully removed using a pipette.
   In contrast, purification by NAP-5 columns includes time for conditioning of the column, elution of different fractions and measurements of the eluted fractions to identify the product fraction(s).

### Example 6

### Drying of Alginate Gel Beads

Dried alginate gel beads added into an aqueous solution have the same cation binding properties as freshly prepared alginate gel beads.

Alginate gel beads can be dried using a freeze dryer or a Speed-Vac concentrator, to name two techniques. The dried alginate gel beads have a shelf life of minimum 1 year when stored in a freezer. Dried alginate gel beads may be used directly or may be allowed to swell for 0.5-1 hour in a suitable solution (for example water, 0.9% NaCl or PBS) before use.

A comparison between fresh and dried strontium cross-linked alginate gel beads were performed using 0.2 g fresh alginate gel beads and 0.2 g (wet weight) Speed-Vac dried alginate gel beads for purification of a ²²⁷Th⁴⁺-solution containing the radioactive daughter nuclides ²²³Ra²⁺, ²¹¹Pb²⁺ and ²¹¹Bi³⁺ (total activity 440 kBq in 1 mL PBS). After incubation at room temperature for 1 hour, the radionuclide binding efficiency was assessed by measuring the amount of each radionuclide found in half the supernatant (½S) and in the rest fraction containing the alginate gel beads (½S+beads). Measurements were conducted on a high performance germanium detector (HPGe, EG&G Ortec GEM 15-P). The fraction of alginate gel binding nuclide was calculated as in Example 4, slightly underestimating the true value, as the volume of the beads is not considered in the calculation. The results are summarized in Table 3, showing very similar results for the removal of ²²³Ra²⁺, ²¹¹Pb²⁺ and ²¹¹Bi³⁺ using fresh and dried alginate gel beads. Hence, fresh and dried alginate gel beads have shown to be equally efficient cation scavengers.

TABLE 2
Fraction captured on fresh or dried alginate beads.

| Sr-alginate | | | |
|---|---|---|---|
| gel beads | ²²³Ra²⁺ | ²¹¹Pb²⁺ | ²¹¹Bi³⁺ |
| Fresh | 97% | 79% | 77% |
| Dried | 96% | 80% | 81% |

### Example 7

### An Improved Method Using Dried Alginate Gel for Purification of Sterile Solutions

An improved purification method using sterile, dried alginate gel beads have been developed to facilitate their use as scavengers of di- and multivalent cations in a sterile production chain. The method is versatile, robust and simple, using standard laboratory equipment and simple handling procedures known to laboratory personnel.

The method consists of a sterile reaction vial containing sterile, dried alginate gel, for example but not limited to vials added alginate gel beads or vials coated with alginate gel. The sterile solution to be purified is added into the vial and incubated for 15-60 minutes at room temperature with gentle mixing, or simply stored in the vial until use. A simple separation of the purified solution and the alginate gel must be performed, of which the choice of separation depends on the type of reaction vial used. Examples of separation techniques are, but not limited to, the following:
1. For sterile containers coated with alginate gel and sealed with a septum, the purified solution may be removed using a sterile syringe coupled to a sterile 0.22 μm syringe filter.
2. For sterile vials with alginate gel beads on a 0.22 μm filter unit, separation may be performed using centrifuge spinning, followed by extraction of the purified, sterile filtrate using a sterile syringe.

As an example, Speed-Vac dried strontium cross-linked alginate gel beads (0.2 g wet weight) was used for purification of a preparation containing a ²²⁷Th-mAb to remove the daughter nuclides ²²³Ra²⁺, ²¹¹Pb²⁺ and ²¹¹Bi³⁺ present in the solution (total activity 2.5 MBq in 0.5 mL PBS). Dried alginate gel beads were placed on the filter of a centrifugal 0.22 μmfilter unit, for example Ultrafree-MC 0.22 μm GV Centrifugal Filter Unit (Millipore, CAS: UFC30GVOS). A preparation of ²²⁷Th-mAb was added into the filter unit, the tube was sealed and the vial incubated at room temperature for 30 minutes with intermittent shaking Purified ²²⁷Th-mAb was separated from the alginate gel beads by centrifuging at 300×g for 3 minutes, followed by washing with 2×0.5 mL PBS. The obtained filtrate is a purified, sterile injection solution of ²²⁷Th-mAb in PBS ready for use.

### LEGENDS TO FIGURES

FIG. 1—Alginate Concentration Gradient

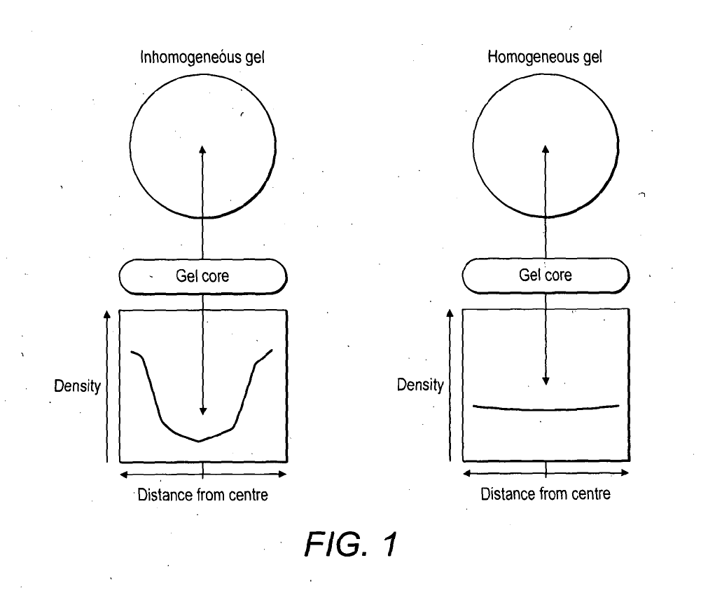

FIG. 2a—Guluronate and Mannuronate units in an alginate polymer

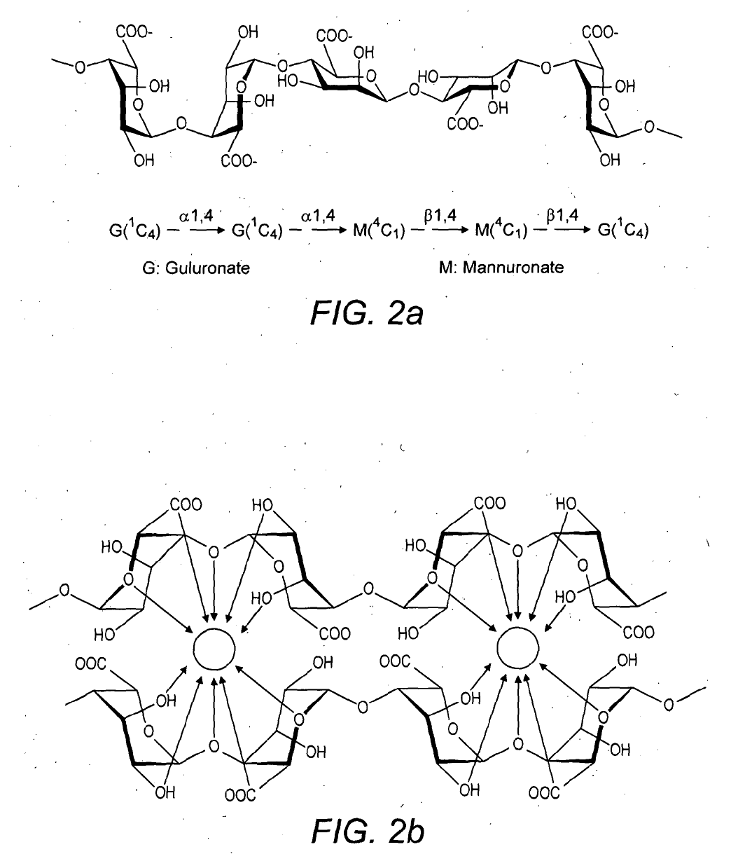

FIG. 2b—Crosslinking of Guluronate subunits in two Alginate chains by a cation

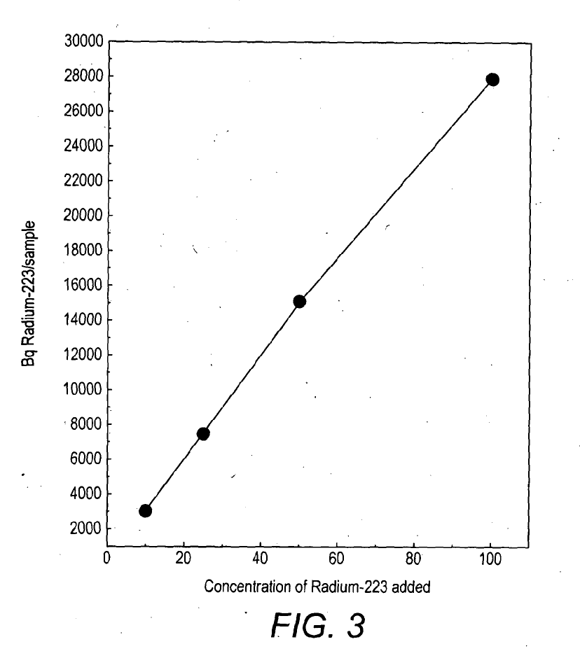

FIG. 3—Radium-223 binding to homogenous Ca-alginate gel beads

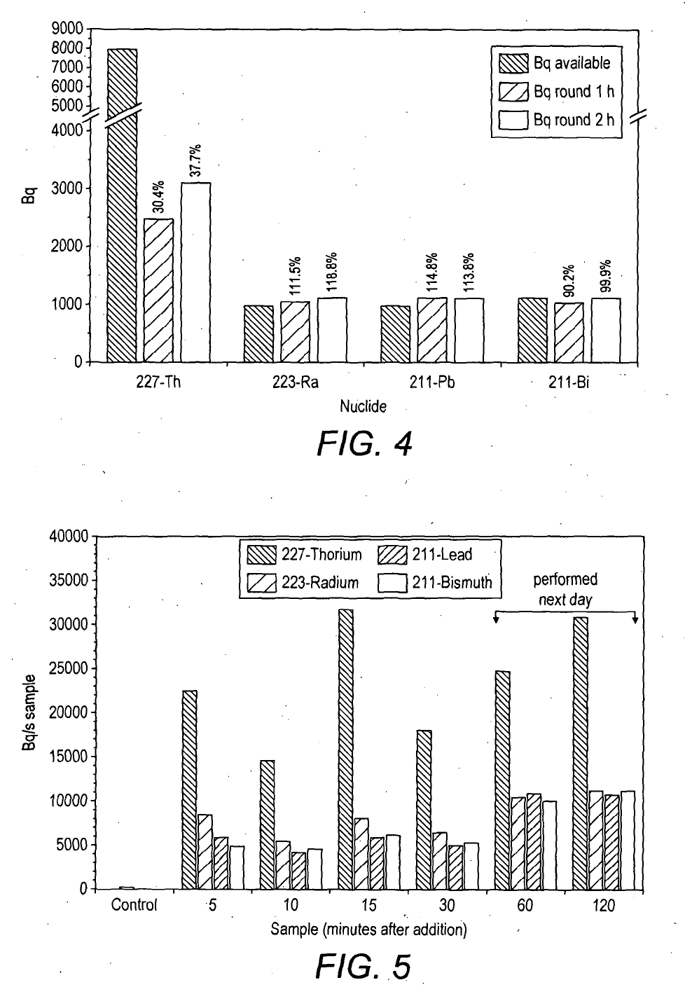

FIG. 4—Binding efficiency of the radionuclides ²²⁷Th⁴⁺, ²²³Ra²⁺, ²¹¹Pb²⁺ and ²¹¹Bi³⁺ to inhomogeneous Ca-alginate gel beads

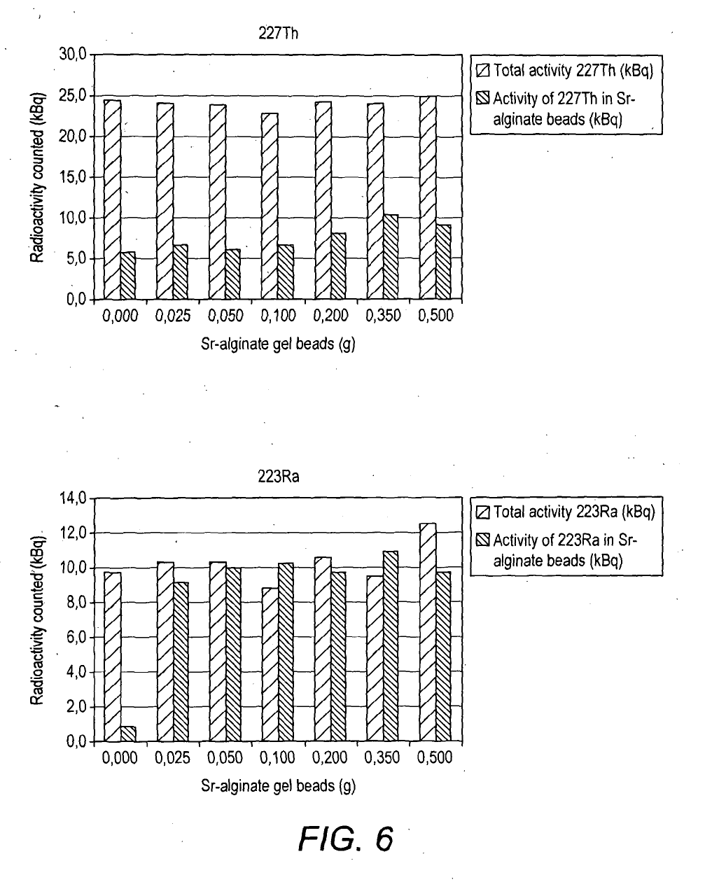

FIG. 5—Binding of ²²⁷Th⁴⁺ to alginate gel beads

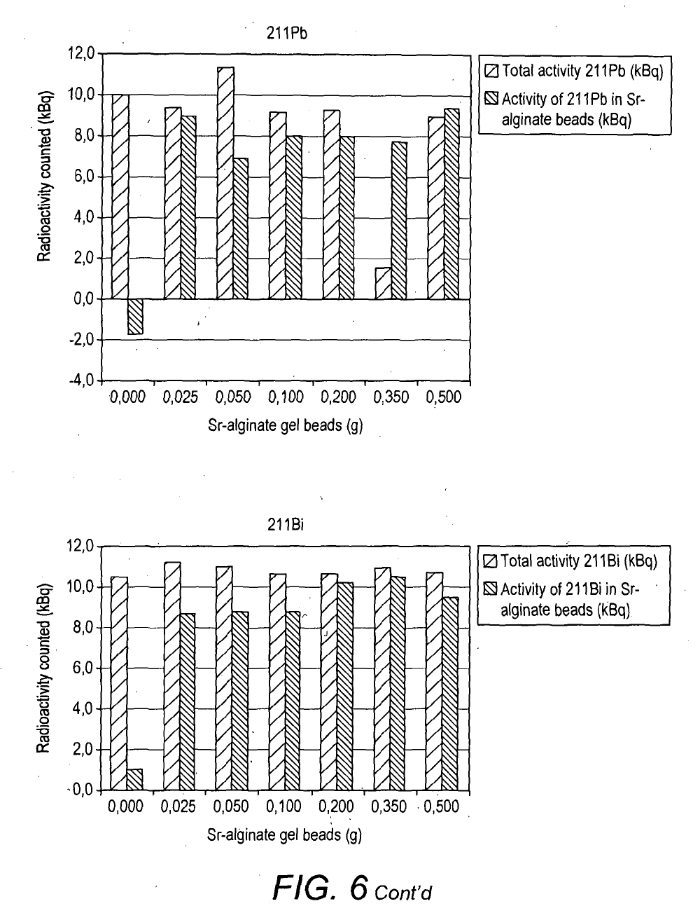

FIG. 6: Purification of a preparation of ²²⁷Th-mAb with daughter nuclides, using 0.000-0.500 g inhomogeneous Sr-alginate gel beads and 1 hour incubation at room temperature.

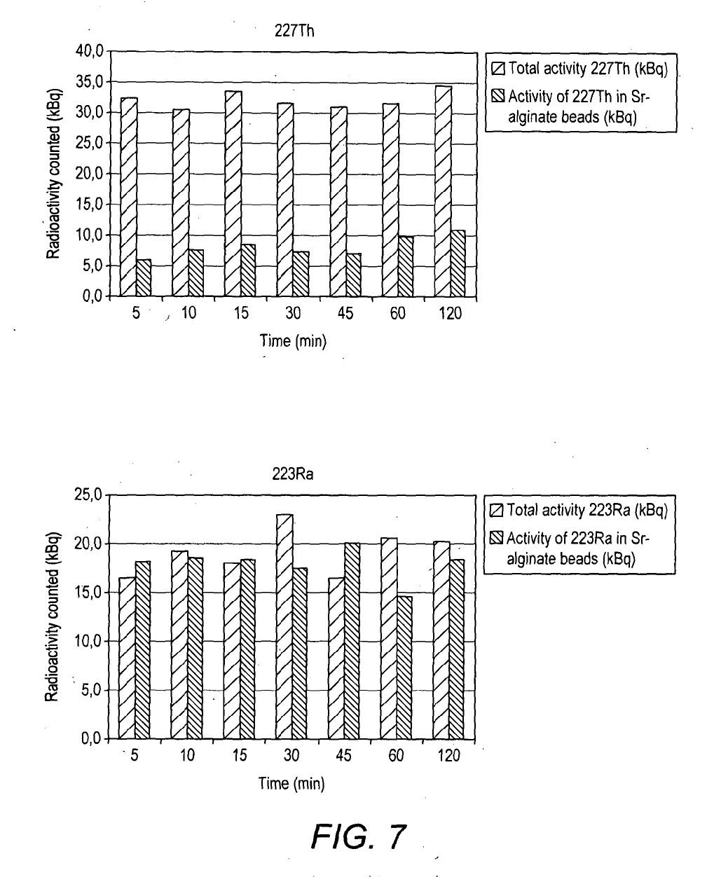

FIG. 7: Purification of a preparation of ²²⁷Th mAb with daughter nuclides, using 0.200 g inhomogeneous Sr-alginate gel beads in each vial and 5-120 min reaction time at room temperature.

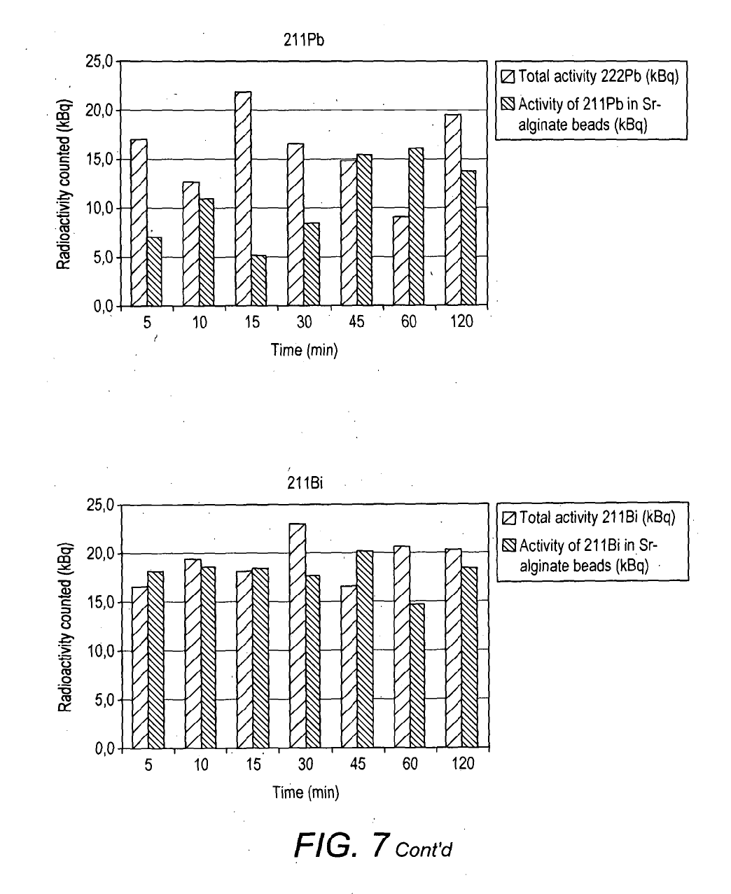

1. A pharmaceutical preparation comprising at least one complexed alpha-emitting radionuclide and at least one polysaccharide biopolymer.

2. A pharmaceutical preparation as claimed in claim 1 wherein said polysaccharide biopolymer absorbs or is capable of absorbing uncomplexed ions.

3. A pharmaceutical preparation as claimed in claim 2 wherein said uncomplexed ions include at least one daughter isotope resulting from the radioactive decay chain of said least one complexed alpha-emitting radionuclide.

4. A pharmaceutical preparation as claimed in claim 1 wherein the solution concentration of uncomplexed ions of alpha emitting radioisotopes contributes no more than 10% of the total count of radioactive decays per unit time.

5. A pharmaceutical preparation as claimed in claim 1 wherein the solution concentration of uncomplexed ions resulting from the radioactive decay chain of said least one complexed alpha-emitting radionuclide is no greater than 10% of the solution concentration of said least one complexed alpha-emitting radionuclide.

6. A pharmaceutical preparation as claimed in claim 1 wherein the proportion of uncomplexed ions resulting from the radioactive decay chain of said least one complexed alpha-emitting radionuclide present in the solution, in comparison with those captured by said at least one polysaccharide biopolymer is no more than 10%.

7. A pharmaceutical preparation as claimed in claim 1 wherein the solution concentration of uncomplexed ions resulting from the radioactive decay chain of said least one complexed alpha-emitting radionuclide contributes no more than 10% of the total count of radioactive decays per unit time from the for a period of at least 4 half-lives of the longest lived of said at least one complexed alpha-emitting radionuclide.

8. A pharmaceutical preparation as claimed in claim 1 wherein said at least one polysaccharide biopolymer comprises at least one alginate.

9. A pharmaceutical preparation as claimed in claim 8 wherein said alginate is in the form of particles such as beads, rods, flakes or sheets; or is present as a coating on a substrate such as a bead or the inside surface of a vessel.

10. A pharmaceutical preparation as claimed in claim 1 wherein said at least one polysaccharide biopolymer is coated with at least one material to maintain its integrity and/or to reduce disintegration due to radiolysis.

11. A pharmaceutical preparation as claimed in claim 1 wherein said at least one complexed alpha-emitting radionuclide is ²²⁷Th, ²²³Ra, or ²²⁵Ac.

12. A pharmaceutical preparation as claimed in claim 1 wherein said at least one complexed alpha-emitting radionuclide is complexed by means of a chelator selected from octadentate hydroxypyridinone-containing chelators, such as those comprising a 1,2-hydroxypyridinone moiety and/or a 3,2-hydroxypyridinone moiety.

13. A pharmaceutical preparation as claimed in claim 1 wherein said at least one complexed alpha-emitting radionuclide is complexed by means of a chelator selected from DOTA (1,4,7,10-tetraazacyclododecane-1,4,7,10-tetraacetic acid) and derivatives of this molecule.

14. A pharmaceutical preparation as claimed in claim 1 wherein said at least one complexed alpha-emitting radionuclide is bound to at least one targeting moiety, such as an antibody, an antibody fragment, a receptor, a receptor binding moiety or a specific binding peptide.

15. A method for generating an injectable solution of at least one complexed alpha-emitting radionuclide, said method comprising contacting a pharmaceutical preparation of said least one complexed alpha-emitting radionuclide with at least one polysaccharide biopolymer and subsequently separating said solution of at least one complexed alpha-emitting radionuclide from said at least one polysaccharide biopolymer.

16. A method as claimed in claim 15 wherein said separation comprises filtration, preferably sterile filtration.

17. A method as claimed in claim 15 wherein said contacting takes place for greater than 50% of the storage period between preparation of said pharmaceutical preparation and separating said solution of at least one complexed alpha-emitting radionuclide from said at least one polysaccharide biopolymer.

18. A method as claimed in claim 15 wherein said contacting takes place for no more than 1 hour prior to separating said solution of at least one complexed alpha-emitting radionuclide from said at least one polysaccharide biopolymer.

19. A method for the removal of at least one uncomplexed radionuclide from a pharmaceutical preparation comprising a solution of at least one complexed alpha-emitting radionuclide, said method comprising contacting said pharmaceutical preparation with at least one polysaccharide biopolymer.

20. A method as claimed in claim 19 further comprising separating said solution from said polysaccharide biopolymer.

21. The use of at least one polysaccharide biopolymer for the removal of at least one uncomplexed radionuclide from a pharmaceutical preparation comprising a solution of at least one complexed alpha-emitting radionuclide.

22. The use as claimed in claim 21 comprising contacting said polysaccharide biopolymer with said pharmaceutical preparation and subsequently separating said polysaccharide biopolymer from said solution.

23. An administration device comprising a pharmaceutical preparation as claimed in claim 1.

24. A kit for the preparation of an injectable solution, said kit comprising least one polysaccharide biopolymer and at least one solution of a complexed alpha-emitting radionuclide.

25. A kit as claimed in claim 24 comprising a pharmaceutical preparation comprising at least one complexed alpha-emitting radionuclide and at least one polysaccharide biopolymer.

26. A kit as claimed in claim 24 additionally comprising a filter and/or an administration device.

27. A kit as claimed in claim 24 comprising a filter of pore size of no larger than 0.22 μm
